(19) United States
(12) Patent Application Publication
(10) Pub. No.: US 2024/0108692 A1
(43) Pub. Date: Apr. 4, 2024

(54) COMBINATION OF AN INSULIN AND A GLP-1-AGONIST

(71) Applicant: Sanofi-Aventis Deutschland GmbH, Frankfurt (DE)

(72) Inventors: Ulrich Werner, Frankfurt am Main (DE); Baerbel Rotthauser, Frankfurt am Main (DE)

(21) Appl. No.: 18/229,793

(22) Filed: Aug. 3, 2023

Related U.S. Application Data
(63) Continuation of application No. 17/237,632, filed on Apr. 22, 2021, now abandoned.
Application No. 17/237,632 is a continuation of application No. 16/165,837, filed on Oct. 19, 2018, now abandoned.
Application No. 16/165,837 is a continuation of application No. 15/340,969, filed on Nov. 1, 2016, now Pat. No. 10,117,909.
Application No. 15/340,969 is a continuation of application No. 13/123,835, filed on Sep. 30, 2011, now Pat. No. 9,526,764.

(30) Foreign Application Priority Data
Oct. 17, 2008 (DE) 10 2008 051 834.4
Oct. 24, 2008 (DE) 10 2008 053 048.4
Aug. 20, 2009 (DE) 10 2009 038 210.0

(51) Int. Cl. A61K 38/28; A61K 38/26

(52) U.S. Cl. CPC A61K 38/28; A61K 38/26

(57) ABSTRACT

The invention relates to a drug comprising at least one insulin and at least one GLP-1 receptor agonist.

The invention relates to a medicament comprising at least one insulin and at least one GLP1 receptor agonist, referred to below as GLP-1 agonist, the medicament being formulated and/or compounded in such a way that it comprises the insulin and the GLP-1 agonist each in a predetermined amount and can be administered in a dose adapted to the individual requirement of a patient.

The invention relates more particularly to a medicament comprising a first pharmaceutical composition and a second pharmaceutical composition, and, optionally, at least one further pharmaceutical composition, which each comprise at least one Insulin and at least one GLP-1 agonist and contain the at least one insulin and/or the at least one GLP-1 agonist in different fractions relative to the total weight of the composition.

More particularly the present invention relates to a medicament which comprises a first pharmaceutical composition and a second pharmaceutical composition, and, optionally, at least one further pharmaceutical composition, the first pharmaceutical composition comprising at least one insulin, and the second pharmaceutical composition comprising at least one insulin and at least one GLP-1 agonist, and the at least one further pharmaceutical composition comprising at least one insulin and at least one further active compound.

Around 250 million people worldwide suffer from diabetes mellitus. For the type 1 diabetics among them, replacement of the deficient endocrine insulin secretion is the only possible therapy at present. Those affected are dependent on insulin injections for life, usually several times a day. Type 2 diabetes contrasts with type 1 diabetes in that there is not always a deficiency of insulin, but in a large number of cases, especially at the advanced stage, treatment with insulin, where appropriate in combination with an oral antidiabetic. Is considered the most advantageous form of therapy.

In healthy individuals, insulin release by the pancreas is strictly coupled to the blood glucose concentration. Elevated blood glucose levels like those occurring after meals are rapidly compensated by a corresponding rise in insulin secretion. In the fasting state, the plasma insulin level falls to a baseline value which is sufficient to ensure a continuous supply of glucose to insulin-sensitive organs and tissues and to keep hepatic glucose production low during the night The replacement of the endogenous insulin secretion by exogenous, usually subcutaneous administration of insulin does not in general come close to the above-described quality of the physiological regulation of blood glucose. Frequently there are instances of blood glucose being thrown off-track, either upwardly or downwardly, and in their most severe forms these may be life-threatening. In addition, however, blood glucose levels which are elevated over years, without initial symptoms, constitute a considerable health risk. The large-scale DCCT study in the USA (The Diabetes Control and Complications Trial Research Group (1993) N. Engl. J. Med. 329, 977-986) showed unambiguously that chronically elevated blood glucose levels are responsible for the development of late diabetic complications. Late diabetic complications are micro- and macrovascular damage which is manifested in certain circumstances as retinopathy, nephropathy or neuropathy and leads to blindness, renal failure, and loss of extremities, and, in addition, is associated with an increased risk of cardiovascular disorders. From this it can be inferred that an improved therapy of diabetes must be aimed primarily at keeping blood glucose as closely as possible within the physiological range, According to the concept of intensified insulin therapy, this is to be achieved by means of injections, several times a day, of fast-acting and slow-acting insulin preparations, Fast-acting formulations are given at mealtimes in order to compensate the postprandial rise in blood glucose. Slow-acting basal insulins are intended to ensure the basic supply of insulin especially during the night, without leading to hypoglycemia.

Insulin is a polypeptide composed of 51 amino acids which are divided over 2 amino acid chains: the A chain, with 21 amino acids, and the B chain, with 30 amino acids. The chains are linked together by 2 disulfide bridges. Insulin preparations have been employed for many years for diabetes therapy. Such preparations use not only naturally occurring insulins but also, more recently, insulin derivatives and insulin analogs.

Insulin analogs are analogs of naturally occurring insulins, namely human Insulin or animal insulins, which differ by replacement of at least one naturally occurring amino acid residue by other amino acids and/or addition/deletion of at least one amino acid residue from the corresponding, otherwise identical, naturally occurring insulin. The amino acids in question may also be amino acids which do not occur naturally.

Insulin derivatives are derivatives of naturally occurring insulin or of an insulin analog which are obtained by chemical modification. The chemical modification may consist, for example, in the addition of one or more defined chemical groups and to one or more amino acids. Generally speaking, the activity of insulin derivatives and insulin analogs is somewhat altered as compared with human insulin.

Insulin analogs with an accelerated onset of action are described in EP 0 214 826, EP 0 375 437, and EP 0 678 522. EP 0 214 826 relates, among other things, to replacements of B27 and B28. EP 0 678 522 describes insulin analogs which have different amino acids in position B29, preferably proline, but not glutamic acid. EP 0 375 437 encompasses insulin analogs with lysine or arginine in B28, which may optionally also be modified in B3 and/or A21. An accelerated activity is also exhibited by the insulin analogs described in EP-A-0 885 961.

EP 0 419 504 discloses insulin analogs which are protected from chemical modifications by modification of asparagine in B3 and of at least one further amino acid in positions A5, A15, A18 or A21.

WO 92/00321 describes Insulin analogs in which at least one amino acid in positions 81-86 has been replaced by lysine or arginine. Such insulins, according to WO 92/00321, have an extended activity. A delayed activity is also exhibited by insulin analogs described in EP-A 0 368 187 and by the insulin analogs described in German patent applications 10 2008 003 568.8 and 10 2008 003 566.1.

The insulin preparations of naturally occurring insulins for insulin replacement that are on the market differ in the origin of the insulin (e.g., bovine, porcine, human insulin) and also in their composition, whereby the profile of action can be influenced (onset of action and duration of action). By combining different insulin products it is possible to obtain a wide variety of profiles of action and to set blood sugar levels which are as close as possible to physiological. Recombinant DNA technology nowadays allows the production of such modified insulins. These include insulin glargine (Gly(A21)-Arg(B31)-Arg(B32) human insulin), with an extended duration of action. Insulin glargine is injected as an acidic, clear solution and, on account of its solution properties in the physiological pH range of the subcutaneous tissue, is precipitated as a stable hexamer associate. Insulin glargine is injected once daily and is notable over other long-activity insulins for its flat serum profile and the associated reduction in the risk of nocturnal hypoglycemias (Schubert-Zsilavecz et al., 2:125-130(2001)).

The specific preparation of insulin glargine that leads to a prolonged duration of action is characterized by a clear solution with an acidic pH.

Glucagon-like peptide 1 (GLP-1) is an endocrine hormone which increases the insulin response following oral intake of glucose or fat. GLP-1 generally regulates the concentrations of glucagons, slows down gastric emptying, stimulates the biosynthesis of (Pro-)insulin, increases the sensitivity toward insulin, and stimulates the insulin-independent biosynthesis of glycogen (Holst (1999), Curr. Med. Chern 6:1005, Nauck et aL (1997) Exp Clin Endocrine! Diabetes 105: 187, Lopez-Delgado et al (1998) Endocrinology 139:2811).

Human GLP-1 has 37 amino acid residues (Heinrich et al., Endocrinol. 115:2176 (1984), Uttenthal et al, J Clin Endocrinol Metabol (1985) 61:472). Active fragments of GLP-1 include GLP-1(7-36) amide and GLP-1(7-37).

Exendins are another group of peptides which are able to lower blood glucose concentrations. Exendins have a certain similarity in sequence to GLP-1(7-36) (53%, Goke et al. J. Biol Chem 268, 19650-55). Exendin-3 and exendin-4 stimulate an increase in cellular cAMP production in acinar cells of the guinea pig pancreas by interaction with exendin receptors (Raufman, 1996, Reg. Peptides 61:1-18). In contrast to exendin-4, exendin-3 produces an increase in amylase release in acinar cells of the pancreas.

Exondin-3, exendin-4, and exendin agonists have been proposed for the treatment of diabetes mellitus and the prevention of hyperglycemia; they reduce gastric motility and gastric emptying (U.S. Pat. No. 5,424,286 and WO98/05351).

Exendin analogs may be characterized by amino acid replacements and/or C.-terminal truncation of the natural exendin-4 sequence. Exendin analogs of this kind are described in WO 99/07404, WO 99/25727, WO 99/25728.

Combinations of insulin and GLP-1 are known from WO 2004/005342 for the treatment of diabetes.

In clinical practice the amount of insulin to be administered is adjusted to the individual requirement of the individual diabetes patients. Individual patients generally need different amounts of insulin and/or GLP-1 agonist. Typically the predetermined dose is administered by administering a defined amount of a composition having a defined concentration. A result of this is that a composition which comprises insulin and GLP-1 at the same time allows the administration of only one particular proportion of insulin and GLP-1. This means that only one of the two amounts of insulin and GLP-1 can be adapted optimally to the requirement of the patients. Since in practice the correct adjustment of the amount of insulin administered is essential, It is assumed that, when a particular proportion of insulin to GLP-1 is administered, the GLP-1 agonist is either underdosed or overdosed and is correct by chance at best

There are various systems known for injecting a combination of active compounds. The active compounds may be formulated in a composition and provided in a device, as for example in a prefilled syringe. A system of this kind does allow the dosing of the combination, but only in a fixed portion of the active compounds, as is present in the composition. As set out therein, this is a disadvantage for the combination of an insulin with a GLP-1 agonist, since different amounts of the insulins and of the GLP-1 agonist have to be administered, according to the therapeutic requirement.

It is also possible for two active compounds to be administered in two separate formulations, each comprising one of the two active compounds, which are injected independently of one another each with one device (e.g., prefilled syringes). In the case of an injection therapy such as the injection of insulin, for example, patient compliance is a key prerequisite for the success of the therapy. Generally speaking, in the case of an injection therapy, pain, needle-phobia, and the carrying facility for the injection apparatus are a problem, which can lead to reduced compliance. If the patient is to use two separate devices for injection, then these problems multiply.

A single device for the administration of insulin and a GLP-1 agonist is advantageous over the use of two separate devices for administering insulin and a GLP-1 agonist as far as the patient/user is concerned. Moreover, the use of only one device rather than two devices may reduce the number of steps which the patient/user must carry out, which lowers the frequency of errors in use. This reduces the risk of unwanted side effects.

U.S. Pat. Nos. 4,689,042, 5,478,323, 5,253,785, and WO 01/02039 describe devices for the simultaneous administration of two injectable products to a patient. These devices comprise two containers each containing one composition. In these devices the two compositions are injected via a needle. This does make it possible to overcome the disadvantages produced by the use of two separate devices. As a result of the mixing process, there is a dilution in the concentrations of the two active compounds. This may impact adversely on the pharmacokinetics.

The pharmacokinetics of insulin, particularly of insulin glargine, is influenced by the dilution of the insulin in the administered composition. In order to ensure reliable activity of a particular dose of insulin, therefore, the concentration of insulin ought to be kept constant as far as possible. Dosing ought to take place essentially via the volume of the insulin composition administered. This is also true for the administration of a combination of insulin and a GLP-1 agonist. When a combination of insulin and a GLP-1 agonist is administered, this proviso can only be met if both substances are dosed in a fixed proportion to one another in one composition. Where both substances are provided in separate compositions and are mixed for injection in a suitable device (e.g., from WO 01/02039), then a constant concentration of insulin can be realized only if the insulin composition is not substantially diluted by the composition of the GLP-1 agonist. This imposes limits on the possibility of independent dosing of insulin and of the GLP-1 agonist.

One conceivable solution would be to provide the GLP-1 agonist in such a high concentration that the dosed addition of the GLP-1 agonist produces not significant dilution of the insulin composition (e.g., not more than 10%). Polypeptides such as insulins (e.g., insulin glargine, Lantus®) or GLP-1 agonists cannot be concentrated ad infinitum. First, the solubility of proteins is limited, and high concentrations of protein may alter the flow characteristics of the solution. The most important problem for the use of solutions with a high concentration of active compound is the dosing accuracy.

At high concentrations it would be necessary to administer small volumes or to carry out dosing into a different solution. There are devices known for the precise dosing of small or very small volumes. However, such devices are expensive and, on the basis of their operation, are intended only for use by trained personnel, as in the laboratory, for example. Since, generally speaking, patients inject themselves with insulins and/or GLP-1 agonists, the use of such devices for administering insulins and/or GLP-1 agonists is ruled out. The devices that are described, for example, In U.S. Pat. Nos. 4,689,042, 5,478,323, 5,253,785, and WO 01/02039, which allow the patients to inject themselves with active compound solutions, are unsuitable for the dosing of small and very small volumes.

The problems which arise with the injection of a combination of an insulin and a GLP-1 agonist are as follows
the proportion of the active compounds must be variable;
the pharmacokinetics of at least one of the active compounds (the insulin) is influenced by the concentration/dilution;
the pharmacokinetics of at least one other active compound (the GLP-1 agonist) is not, or not substantially, influenced by the concentration/dilution.

It was an object of the present invention, therefore, to provide a medicament which at least partly overcomes the above-described disadvantages of the prior art. A further intention is that there should as far as possible be only one administration per day.

It has been found, surprisingly, that the combination of an insulin with a GLP-1 agonist exhibits synergistic effects in the regulation of blood glucose in the postprandial and postabsorptive phases as compared with the use of insulin or the GLP-1 agonist alone:
Higher activity on the basis of the combination of the complementary activities on the fasting and postprandial glucose levels, which complement one another (examples 2 and 3). The combination exhibits a lowering in postprandial glucose concentration (i.e., improved glucose tolerance) like a GLP-1 agonist alone, and additionally exhibits the postabsorptive lowering of glucose like an insulin (example 9).
Reduction in the risk of hypoglycemia (examples 2-4).
Improved adaptation of the blood glucose concentration to normoglycemic levels (example 8).
Improved glucose tolerance and lowering of postabsorptive glucose concentrations (example 9).
The synergistic activities of the combination on the glucose concentration are observed in a GLP-1 agonist concentration range of one order of magnitude (factor 10). (Example 6 compared with examples 4 and 2). Only in the case of relatively small GLP-1 doses and/or relatively large proportions of insulin to GLP-1 are the activities of insulin predominant.
Maintains the function of the β-cells (example 10).
Weight loss/reduction in weight gain.
All examples show that GLP-1 agonists and insulins exhibit no adverse interactions.
As a result of the activities on the fasting, postprandial, and postabsorptive blood glucose concentrations, it becomes possible to reduce the number of administrations of the combination to once daily.

The invention provides a medicament comprising at least one insulin and at least one GLP-1 agonist, the medicament being formulated and/or compounded in such a way that it comprises the insulin and the GLP-1 agonist each in a predetermined amount and can be administered in a dose adapted to the individual requirement of a patient.

The medicament of the invention is used in particular for treating patients with diabetes mellitus, more particularly patients with type 1 or type 2 diabetes.

The medicament of the invention allows the blood glucose concentration to be adapted more effectively to normoglycemic levels in the case of patients with diabetes, more particularly type 1 or type 2 diabetes.

The medicament is used preferably to adjust the fasting, postprandial and/or postabsorptive blood glucose concentration of patients with diabetes, more particularly patients with type 1 or type 2 diabetes. More preferably the medicament of the invention is used to adjust the postprandial and/or postabsorptive blood glucose concentration of patients with diabetes, more particularly patients with type 1 or type 2 diabetes. Adjustment in this context means that normoglycemlc blood glucose concentrations are substantially achieved or at least an approximation thereto is obtained. By normoglycemic levels are meant more particularly blood glucose concentrations in the normal range (breadth of fluctuation 60-140 mg/dl, corresponding to 3.3 to 7.8 mmol/1). This range of fluctuation encompasses blood glucose concentrations under fasting conditions, postprandial conditions, and postabsorptive conditions.

Postprandial and postabsorptive are terms familiar to the person skilled in the field of diabetology. Postprandial is used herein to refer more particularly to the phase after a meal and/or after glucose loading in an experiment. This phase is characterized more particularly in a healthy individual by an increase and fall again in the concentration of glucose in the blood. Postabsorptive, or postabsorptive phase, is used herein to refer more particularly to the phase which follows the postprandial phase. The postprandial phase typically ends up to 4 h after the meal and/or glucose loading. The postabsorptive phase lasts typically for up to 8 to 16 h.

The medicament of the invention is also used preferably for improving glucose tolerance in the treatment of a patient with diabetes, more particularly with a type 1 or type 2 diabetes. Improving the glucose tolerance means that the medicament of the invention lowers the postprandial blood glucose concentration. Improving the glucose tolerance is also taken to mean that the medicament of the invention lowers the postabsorptive blood glucose concentration. Lowering means more particularly that the blood glucose concentration substantially reaches normoglycemic values or at least is approximated thereto.

The medicament of the invention is able to lower the risk of hypoglycemia, which may occur, for example, in the postabsorptive phase. The medicament of the invention is used preferably for preventing hypoglycemia in the treatment of a patient with diabetes, more particularly with a type 1 or type 2 diabetes, it being possible for the hypoglycemia to occur more particular in the postabsorptive phase.

The medicament of the invention may maintain the function of the pancreatic β-cells. The medicament of the invention is used preferably for preventing a loss of function of the pancreatic β-cells in a patient with diabetes, more particularly with a type 1 or type 2 diabetes. The loss of function of the β-cells may be caused more particularly by apoptosis.

Furthermore, the medicament of the invention may bring about a loss in weight and/or prevent a gain in weight in patients with diabetes, more particularly type I or II. In diabetes patients, especially those of type 2, weight gain and excessive weight are frequent problems. Accordingly, administering the medicament of the invention may support a therapy for the treatment of excessive weight.

It will be appreciated that the medicament of the invention can be used in order to treat more than one of the preferred indications described therein in a patient with diabetes, more particularly with a type 1 or 2 diabetes. Accordingly the present invention encompasses not only the individual preferred indications but also arbitrary combinations of the indications. The medicament of the invention can therefore be used to treat one or more of the herein-described indications in patients with diabetes, more particularly of patients with type 1 or type 2 diabetes, for the purpose, for example, of adjusting the fasting, postprandial and/or postabsorptive blood glucose concentration, for improving glucose tolerance, for preventing hypoglycemia, for preventing a loss of function of the pancreatic β-cells, for weight loss and/or for preventing weight gain, Preference is given to the adjustment of fasting, postprandial and/or postabsorptive blood glucose concentration, the improvement of glucose tolerance and/or the prevention of hypoglycemia.

The medicament of the invention can also be used for producing a medicinal product for treating one or more of the herein-described indications, as, for example, for adjusting the fasting, postprandial and/or postabsorptive blood glucose concentration, for improving glucose tolerance, for preventing hypoglycemia, for preventing a loss of function of the pancreatic β-cells, for weight loss and/or for preventing weight gain.

The at least one insulin and the at least one GLP-1 agonist may also be used for producing a medicinal product for treating one or more of the herein described indications, as for example for adjusting the fasting, postprandial and/or postabsorptive blood glucose concentration, for improving glucose tolerance, for preventing hypoglycemia, for preventing a loss of function of the pancreatic β-cells, for weight loss and/or for preventing weight gain.

The at least one GLP-1 agonist and the at least one insulin may be provided together in one pharmaceutical composition. In this case a first and a second composition and, optionally, at least one further pharmaceutical composition are provided, each comprising the insulin and the GLP1 agonist. Accordingly the invention provides a medicament comprising a first pharmaceutical composition and a second pharmaceutical composition, and, optionally, at least one further pharmaceutical composition, each comprising at least one insulin and at least one GLP-1 agonist, and containing the at least one insulin and/or the at least one GLP-1 agonist in different weight fractions relative to the total weight of the composition.

In the present specification “optionally, at least one further pharmaceutical composition” means that the medicament of the invention, in addition to the first and second pharmaceutical compositions, may comprise at least one further pharmaceutical composition. Hence the medicament of the invention may comprise, for example, 3, 4, 5, 6, 7, 8, 9, 10 or more pharmaceutical compositions of the invention.

Preferred medicaments are those which comprise a first and a second pharmaceutical composition of the invention.

Likewise preferred are medicaments which comprise a first, a second, and a third pharmaceutical composition of the invention.

Likewise preferred are medicaments which comprise a first, a second, a third, and a fourth pharmaceutical composition of the invention.

Likewise preferred are medicaments which comprise a first, a second, a third, a fourth, and a fifth pharmaceutical composition.

The weight fractions of the at least one insulin and of the at least one GLP-1 agonist may be selected in the first pharmaceutical composition, the second pharmaceutical composition, and, where used, the at least one further pharmaceutical composition in such a way that the pharmaceutical compositions contain different proportions of insulin to GLP-1 agonist, based on the weight fraction.

In this case the first composition may contain the smallest proportion and the second composition the next-greater proportion. Where at least one further composition is present, it may contain the next-greater proportion. Where a further composition is present as well, it may contain the next-greater proportion in turn. The compositions may therefore contain proportions of insulin to GLP-1 agonist, based on the weight fraction, that increase from the first to the second and, where used, further compositions.

The weight fraction of one of the two active compounds, i.e., of the at least one insulin or of the at least one GLP-1 agonist, in the first pharmaceutical composition, the second pharmaceutical composition, and, where used, the at least one further pharmaceutical composition is preferably selected in each case such that the predetermined dose of this active compound can be administered by administering a defined volume of the first, second and/or at least one further composition. With particular preference this active compound is the at least one insulin.

The weight fraction of the other of the two active compounds, i.e., of the at least one insulin or of the at least one GLP-1 agonist, in the first pharmaceutical composition, the second pharmaceutical composition, and, where used, the at least one further pharmaceutical composition is preferably selected such that the proportions of insulin to GLP-1 agonist, based on the weight fraction, increase from the first to the second and, where used, further compositions. With particular preference this active compound is the at least one GLP-1 agonist.

Furthermore, the weight fraction of the other of the two active compounds in the pharmaceutical compositions is determined such that one of the pharmaceutical compositions can be selected in such a way that the dose of the first of the two active compounds that is to be administered and the dose of the second active compound that is to be administered are given in a defined volume. Hence a pharmaceutical composition is selected which contains the desired proportion.

Theoretically it would be possible to provide a pharmaceutical composition for each individual therapeutically desired proportion of the weight fractions of the at least one insulin to the at least one GLP-1 agonist, in order to obtain an optimum dosage, tailored to requirements, for both active compounds for every patient.

In the present invention, a particular number of pharmaceutical compositions is sufficient in order to cover the dosages needed in practice for the two active compounds. For each patient a defined dosage range is defined within a therapeutically rational interval for each of the two active compounds. The dose to be administered ought hereby to fluctuate essentially within this dosage range for a particular patient, without any overdosing or underdosing.

Surprisingly it has been found that the synergistic effects of the combination of at least one insulin and at least one GLP-1 agonist on the concentration of glucose in the blood plasma occur in a concentration range of the GLP-1 agonist of one order of magnitude (factor 10). Since it is primarily the amount of insulin that must be adapted and precisely dosed to the individual patient, the synergistic concentration range of the GLP-1 agonist allows a pharmaceutical composition of the invention that contains a defined proportion of at least one insulin to the at least one GLP-1 agonist to cover a therapeutic range of insulin doses simultaneously with the associated, synergistic amount of GLP-1 agonist. The proportion can be selected such that every desired insulin dose has its corresponding dose of the at least one GLP-1 agonist, which is situated within the desired range, e.g., the synergistic range. As set out earlier on above, the proportions of the first, second, and, where used, at least one further composition of the medicament may also be chosen such that the proportions increase from the first to the second and, where used, the at least one further composition. If the dose of the GLP-1 agonist at the desired insulin dose of a composition (e.g., of the first composition) is outside (generally above) the desired dosage range of the GLP-1 agonist, then the next composition (e.g., the second composition) or a further composition with a greater proportion of the at least one insulin to the at least one GLP-1 agonist, is selected for use, in which the amount of the GLP-1 agonist at the desired insulin dose lies within the desired range. The proportions of the first, second, and, where used, at least one further composition of the medicament may further be chosen such that the ranges of the insulin dosages which correspond to the desired dosages of the at least one GLP-1 agonist border one another and/or overlap one another. Preferably the ranges overlap. Overlapping means more particularly that it is possible to select at least two compositions which, at the desired dose of the at least one insulin, each contain an amount of the at least one GLP-1 agonist which lies within the desired dosage range.

For example, three compositions are sufficient to adjust the dose of the at least one insulin for an individual patient to a level selected from the range from 15 to 80 units of insulin and at the same time to dose the GLP-1 agonist with an amount within the range from 10 to 20 μg (see example 11).

It is also possible to provide a medicament of the invention in which the proportion is selected such that for each desired dosage of the GLP-1 agonist there is a corresponding dosage of the at least one insulin which lies within the desired range, e.g., the synergistic range. The proportions of the first, second, and, where used, at least one further composition of the medicament may also be chosen such that the ranges of the dosages of the GLP-1 agonist that correspond to the desired dosages of the at least one insulin border one another and/or overlap one another. Preferably the ranges overlap. Overlapping in this context means more particularly that it is possible to select at least two compositions which, at the desired dosage of the at least one GLP1 agonist, each contain an amount of the at least one insulin that lies within the desired dosage range.

Preferably the medicament of the invention contains not more than 10 pharmaceutical compositions as defined above, more preferably not more than 5, not more than 4, not more than 3 or 2 pharmaceutical compositions.

The compositions of the invention may contain the at least one insulin in identical or different weight fractions. For example, at least two of the compositions of the invention may contain the at least one insulin in a substantially identical weight fraction.

It is preferred for the first, second, and, where used, further compositions to contain the at least one insulin in a substantially identical weight fraction and the at least one GLP-1 agonist in different weight fractions.

The compositions of the invention may contain the at least one GLP-1 agonist in identical or different weight fractions. For example, at least two of the compositions of the invention may contain the at least one GLP-1 agonist in a substantially identical weight fraction.

It is also preferred for the first, second, and, where used, further compositions to contain the at least one GLP-1 agonist in a substantially identical weight fraction and the at least one Insulin in different weight fractions.

Besides the first, second, and, where used, at least one further composition, the medicament of the invention may comprise at least one further pharmaceutical composition which contains either at least one insulin or at least one GLP-1 agonist.

The medicament of the invention may also comprise at least one further pharmaceutical composition which contains at least one insulin and at least one GLP-1 agonist in a proportion of the weight fractions which is like the herein-described first, second or, where used, further pharmaceutical composition.

The invention further provides a medicament comprising a first pharmaceutical composition and a second pharmaceutical composition, the first pharmaceutical composition comprising at least one insulin and the second pharmaceutical composition comprising at least one GLP-1 agonist, the medicament being formulated and/or compounded for the independent administration of the first and second pharmaceutical compositions.

Example 12 shows how a combination of two or more active compounds can be formulated such that, when two or more compositions are combined, both active compounds can be administered in any desired amounts and in any desired proportions to one another. This takes account of the fact that at least one of the active compounds must not be diluted as a result of the combining (e.g., through mixing immediately prior to administration).

The present invention provides a medicament which comprises a first active compound and a second active compound, and, optionally, at least one further active compound, these active compounds being provided in a first, a second, and, optionally, at least one further composition. The first active compound is present in all of the compositions. The second active compound is present in the second formulation, and the at least one further active compound, where used, is present in the optionally at least one further composition. Hence the second and each further composition comprise the first active compound in combination with another active compound.

The present invention thus further provides a medicament which comprises a first pharmaceutical composition and a second pharmaceutical composition, and, optionally, at least one further pharmaceutical composition, the first pharmaceutical composition comprising at least one first active compound, and the second pharmaceutical composition comprising at least one first active compound and at least one second active compound, and the at least one further pharmaceutical composition comprising at least one first and at least one further active compound. The active compounds here may be any desired active compounds.

The first composition preferably comprises as active compound only the at least one first active compound.

The first, second, and, where used, at least one further compositions may comprise the first active compound in a substantially identical weight fraction or In different weight fractions relative to the total weight of the composition.

It is preferred for the first pharmaceutical composition, the second pharmaceutical composition, and, where used, the at least one further pharmaceutical composition to comprise the first active compound in substantially equal weight fractions relative to the total weight of the composition. By this means it is possible to ensure that any desired proportion of the first and second composition and, where appropriate, any desired proportion of the first and at least one further composition can be used, the dosing of the first active compound taking place via the total amount of the compositions administered. Via the proportion of the two compositions it is possible to increment steplessly the amount of the active compound which is present only in the second composition and, where appropriate, in the at least one further composition. In this way, therefore, it is readily possible to dose any desired amount and any desired proportion of the first to the second active compound and, where appropriate, of the first active compound to a further active compound, without altering the concentration of the first active compound.

The first active compound may be at least one insulin. The second active compound may be at least one GLP-1 agonist. Preference is given to a medicament which comprises a first pharmaceutical composition and a second pharmaceutical composition, and, optionally, At least one further pharmaceutical composition, the first pharmaceutical composition comprising at least one insulin, and the second pharmaceutical composition comprising at least one insulin and at least one GLP-1 agonist, and the at least one further pharmaceutical composition comprising at least one insulin and at least one further active compound.

The first composition preferably comprises as active compound only the at least one insulin.

The further active compound may be any desired active compound. More particularly the further active compound is an active compound which is used for treating patients with diabetes mellitus (type 1 and/or type 2), including active compounds for treating concomitant disorders of diabetes as well.

The first, second, and, where used, at least one further composition may comprise the insulin in a substantially equal weight fraction or in different weight fractions relative to the total weight of the composition.

It is preferred for the first pharmaceutical composition, the second pharmaceutical composition, and, where used, the at least one further pharmaceutical composition to comprise the insulin in substantially equal weight fractions relative to the total weight of the composition. By this means it is possible to ensure that any desired proportion of the first and second composition and, where appropriate, any desired proportion of the first and at least one further compositions can be used, the dosing of the insulin taking place via the total amount of the compositions administered. Via the proportion of the two compositions it is possible to increment steplessly the amount of the active compound which is present only in the second composition and, where appropriate, in the at least one further composition. In this way, therefore, it is readily possible to dose any desired amount and any desired proportion of insulin to GLP-1 agonist and, where appropriate, of insulin to a further active compound, without altering the concentration of the at least one insulin.

In the present invention, “substantially equal weight fractions” of an active compound in two compositions means that one of the two compositions contains the active compound in a weight fraction which is, for example, not more than 10%, not more than 5%, not more than 1% or not more than 0.1% higher than its weight fraction in the other composition.

The first active compound may also be at least one GLP-1 agonist. The second active compound may be at least one insulin. Preference is given to a medicament which comprises a first pharmaceutical composition and a second pharmaceutical composition, and, optionally, at least one further pharmaceutical composition, the first pharmaceutical composition comprising at least one GLP-1 agonist, and the second pharmaceutical composition comprising at least one GLP-1 agonist and at least one insulin, and the at least one further pharmaceutical composition comprising at least one GLP-1 agonist and at least one further active compound.

The first composition preferably comprises as active compound only the at least one GLP1 agonist.

The first, second, and, where used, at least one further compositions may comprise the GLP-1 agonist in a substantially equal weight fraction or in different weight fractions relative to the total weight of the composition. It is preferred for the first pharmaceutical composition, the second pharmaceutical composition and, where used, the at least one further pharmaceutical composition to comprise the at least one GLP-1 agonist in substantially equal weight fractions relative to the total weight of the composition.

Accordingly the present invention provides a medicament which exhibits a number of advantages over compositions of the prior art comprising separate compositions each containing one active compound, more particularly an insulin or a GLP-1 agonist, said advantages including the following:
the ratio of the first active compound to the second active compound and, where appropriate, of the first active compound to the at least one further active compound can be chosen freely by the user.
Since the first active compound is present in all of the compositions, more particularly in equal weight fractions, this active compound is not diluted when the first composition is mixed with the second and, where appropriate, further compositions. This is important for active compounds such as insulin, for example, where the pharmacokinetics is influenced by the concentration/dilution.
The injection volume is reduced (see example 12). Hence there is a reduction in the dilution of the second active compound (e.g., a GLP-1 agonist) and, where appropriate, of a further active compound.

The invention additionally provides a kit comprising a medicament of the invention. The kit of the invention may be intended for use by medical staff or by persons without specialist medical training, more particularly by the patients themselves or helpers such as relatives. In the kit of the invention, the individual pharmaceutical compositions comprising the medicament of the invention are assembled in separate packs, and so the patient is able to select the composition appropriate to the current requirement and to administer an amount in line with that requirement. The kit of the invention comprises, for example, the medicament of the invention in the form of a set of syringes, glass ampoules and/or pens which comprise a composition of the invention.

There are a variety of ways in which the medicament of the invention can be administered. The medicament may be administered parenterally. The medicament may be injected, with the possible use of injection systems with or without needles. Furthermore, the medicament may be administered by inhalation. In this case it is possible for liquid compositions to be inhaled, or the compositions can be inhaled in the form of powder. Furthermore, the medicament of the invention may be administered as a spray, more particularly as a nasal spray. In addition the medicament of the invention may be administered by a transdermal system. The skilled worker is aware of these methods of administration and is able to formulate the medicament of the invention in such a way that it can be effectively administered by one of these methods of administration. The compositions of the medicament of the invention are preferably liquid. It is preferred, furthermore, for the medicament of the invention to be administered parenterally, more particularly by injection.

The present invention further provides a device for administering the medicament of the invention. This device comprises the pharmaceutical compositions which are encompassed by the medicament of the invention, in separate containers, and allows the pharmaceutical compositions to be dosed independently of one another. The device of the invention may be a device for parenteral administration. The device of the invention may be a device for injection with or without needles. Furthermore, the device may be a device for inhalation, in which case liquid compositions are inhaled, or the compositions can be inhaled in the form of powder. Moreover, the device may be a device for administering a spray, more particularly a nasal spray. In addition, the device may be a transdermal administration system. It is preferred for the device of the invention to be a device for parenteral administration, more particularly an injection device.

“Compounding” is a term which is known to the skilled worker and which in pharmacology identifies the finishing treatment, such as portioning and packing, for example, of medicaments for use by the end user. In the present specification, “compounded” or “compounding” means more particularly that the pharmaceutical compositions of the invention are packaged in a suitable way in a therapeutically effective amount to allow the herein-described selection of at least one of the compositions of the medicament of the invention for the desired dosing of the at least one insulin and of the at least one GLP-1 agonist. Intended more particularly is a parenteral administration, preferably an injection, more preferably subcutaneous injection. Suitable packaging is, for example, a syringe or a glass vessel with a suitable closure, from which, as required, individual therapeutically active doses can be taken. Likewise suitable are injection pens for the administration of insulin, comprising a container (e.g., a cartridge) which contains a pharmaceutical composition of the invention.

“Formulating” or “formulation” is a term which is known to the skilled worker and which, in the field of pharmacology, refers to the production of medicaments and medicament compositions, and their preparation with excipients. In the present specification “formulating” or “formulation” means more particularly that the composition of the invention is provided in a suitable form which allows administration of a therapeutically effective amount of the active compounds. More particularly a formulation is intended for parenteral administration, preferably for injection, more preferably for subcutaneous injection.

In the present invention the term “GLP-1 agonist” includes GLP-1, analogs and derivatives thereof, exendin-3 and analogs and derivatives thereof, and exendin-4 and analogs and derivatives thereof. The compositions of the invention comprise one or more selected independently of one another from the group consisting of glucagon-like peptide-1 (GLP-1), analogs and derivatives of GLP-1, exendin-3, analogs and derivatives of exendin-3, exendin-4, analogs and derivatives of exendin-4, and pharmacologically tolerable salts thereof. Also included are substances which exhibit the biological activity of GLP-1.

GLP-1 analogs and derivatives are described in WO 98/08871, for example; exendin-3, analogs and derivatives of exendin-3, and exendin-4 and analogs and derivatives of exendin-4 can be found in WO 01/04156, WO 98/30231, U.S. Pat. No. 5,424,286, in EP application 99 610043.4, in WO 2004/005342 and WO 04/035623. These documents are included herein by reference. The exendin-3 and exendin-4 described in these documents, and the analogs and derivatives thereof that are described there, can be used in the compositions of the present invention as GLP-1 agonists. It is also possible to use any desired combinations of the exendin-3 and exendin-4 described in these documents, and the analogs and derivatives described therein, as GLP-1 agonists, The at least one GLP-1 agonist is preferably independently selected from the group consisting of exendin-4, analogs and derivatives of exendin-4, and pharmacologically tolerable salts thereof.

A further preferred GLP-1 agonist is an analog of exendin-4 selected from a group consisting of:
H-desPro³⁶-exendin-4-Lys₆-NH₂,
H-des(Pro^{36,37})-exendin-4-Lys₄NH₂,
H-des(Pro^{36,37})-exendin-4-Lys₅-NH₂, and pharmacologically tolerable salts thereof.

A further preferred GLP-1 agonist is an analog of exendin-4 selected from a group consisting of:
desPro³⁶ [Asp²⁸]exendin-4 (1-39),
desPro³⁶ [IsoAsp²⁸]exendin-4 (1-39),
desPro³⁶ [Met(O)¹⁴, Asp²⁶]exendin-4 (1-39),
desPro¹⁶ [Met(O)¹⁴, IsoAsp²⁸]exendin-4 (1-39),
desPro³⁶ [Trp(O₂)²⁵, Asp²⁸]exendin-2 (1-39),
desPro³⁶ [Trp(O₂)²⁵, IsoAsp²⁸]exendin-2 (1-39),
desPro³⁶ [Met(O)¹⁴Trp(O₂)²⁵, Asp²⁸]exendin-4 (1-39),
desPro³⁶ [Met(O)¹⁴Trp(O₂)²⁵, IsoAsp²⁸]exendin-4 (1-39), and pharmacologically tolerable salts thereof.

A further preferred GLP-1 agonist is an analog of exendin-4 selected from a group as described in the paragraph above in which the peptide -Lys₆-NH₂ has been attached at the C-terminii of the analogs of exendin-4.

A further preferred GLP-1 agonist is an analog of exendin-4 selected from a group consisting of:
H-(Lys)₆-des Pro³⁶ [Asp²⁸]exendin-4(1-39)-Lys₆-NH₂
des Asp²⁸Pro³⁶, Pro³⁷, Pro₃₈ exendin-4(1-39)-NH₂,
H-(Lys)₆-des Pro³⁶, Pro³⁷, Pro³⁸ [Asp²⁸]exendin-4(1-39)-NH₂,
H-Asn-(Glu)₅ des Pro³⁶, Pro³⁷, Pro³⁸ [Asp²⁸]exendin-4(1-39)-NH₂,
des Pro³⁶, Pro³⁷, Pro³⁸ [Asp²⁸]exendin-4(1-39)-(Lys)₆-NH₂,
H-(Lys)₆-des Pro³⁶, Pro³⁷, Pro³⁸ [Asp²⁸]exendin-4(1-39)-(Lys)₆-NH₂,
H-Asn-(Glu)-des Pro³⁶, P Pro³⁸ [Asp²⁸]exendin-4(1-39)-(Lys)₆-NH₂,
H-(Lys)6-des Pro³⁶[Trp(O₂)₂, Asp²⁸]exendin-4(1-39)-Lys₆-NH₂,
H-des Asp⁸ Pro³⁶, Pro³⁷, Pro³⁸ [Trp(O₂)²⁵]exendin-4(1-39)-NH₂,
H-(Lys)₆ des Pro³⁶, Pro³⁷, Pro³⁸ [Trp(O₂)²⁵, Asp]exendin-4(1-39)-NH₂,
H-Asn-(Glu)-des Pro³⁶, Pro³⁷, Pro³⁸ [Trp(O₂)²⁵, Asp²⁶]exendin-4(1-39)-NH₂,
des Pro³⁶, Pro³⁷, Pro³⁸ [Trp(O₂)²⁵, Asp²⁸]exendin-4(1-39)-(Lys)₆-NH₂,
H-(Lys)₆ des Pro³⁶, Pro³⁷, Pro³⁸[Trp(O₂)²⁵, Asp²⁸]exendin-4(1-39)-(Lys)₆-NH₂,
H-Asn-(Glu)₅ des Pro³⁶, Pro³⁷, Pro³⁸ [Trp(O₂)²⁵, Asp²⁸]exendin-4(1-39)-(Lys)₆-NH₂,
H-(Lys)₆-des Pro³⁶ [Met(O)¹⁴, Asp²⁸]exendin-4(1-39)-Lys₆-NH₂,
des Met(O)¹⁴ Asp²⁸ Pro³⁶, Prop³⁷, Pro³⁸ exendin-4(1-39)-NH₂,
H-(Lys)₆-des Pro³⁶, Pro³⁷, Pro³⁸ [Met(O)¹⁴, Asp²⁸]exendin-4(1-39)-NH₂,
H-Asn-(Glu)₅-des Pro³⁶, Pro³⁷, Pro³⁸ [Met(O)¹⁴, Asp²⁸]exendin-4(1-39)-NH₂,
des Pro³⁶, Pro³⁷, Pro³⁸ [Met(O)¹⁴, Asp²⁸]exendin-4(1-39)-(Lys)₆-NH₂,
H-(Lys)₆-des Pro³⁶, Pro³⁷, Pro³⁸[Met(O)¹⁴, Asp²⁸]exendin-4(1-39)-Lyse-NH₂,
H-Asn-(Glu)₅ des Pro³⁸, Pro³⁷, Pro³⁸[Met(O)¹⁴, Asp²⁸]exendin-4(1-39)-(Lys)₆-NH₂,
H-(Lys)₆-des Pro³⁸ [Met(O)¹⁴, Trp(O₂)²⁸, Asp²⁸]exendin-4(1-39)-Lyse-NH₂,
des Asp²⁸ Pro³⁶, Pro³⁷, Pro³⁸ [Met(O)¹⁴, Trp(O₂)²⁵]exendin-4(1-39)-NH₂,
H-(Lys)₆-des Pro³⁶ Pro³⁷, Pro³⁸ [Met(O)¹⁴, Trp(O₂)²⁵, Asp²⁸]exendin-4(1-39)-NH₂,
H-Asn-(Glu)₅-des Pro³⁶, Pro³⁷, Pro³⁸ [Met(O)¹⁴, Asp²⁸]exendin-4(1-39)-NH₂,
des Pro³⁶, Prop³⁷, Pro³⁸ [Met(O)⁴, Trp(O₂)¹⁴, Asp²⁸]exendin-4(1-39)-(Lys)₆-NH₂,
H-(Lys)₆-des Pro³⁶, Pro³⁷, Pro³⁸ [Met(O)¹⁴, Trp(O₂)¹⁴, Asp²⁸]exendin-4(1-39)-(Lys)₆-NH₂,
H-Asn-(Glu)₅-des Pro³⁶, Pro³⁷, Pro³⁶ [Met(O)¹⁴, Trp(O₂)²⁵, Asp²⁸]exendin-4(1-39)-(Lys)₆-NH₂, and pharmacologically tolerable salts thereof.

A further preferred GLP-1 agonist is selected from a group consisting of Arg³⁴,Lys²⁶(N^{E}(γ-glutamyl(N^{α}-hexadecanoyl)))GLP-1(7-37) [liraglutide] and a pharmacologically tolerable salt thereof.

A further preferred GLP-1 agonist is AVE0010. AVE0010 has the sequence of Pro³⁸exendin-4(1-39)-Lyse-NH₂. This substance is published as SEQ ID No: 93 in WO 01/04156. Preference is also given to pharmacologically tolerable salts of AVE0010.

The term “at least one GLP-1 agonist” includes combinations of the herein-described GLP-1 agonists which are used in the compositions of the invention, examples being any desired combinations of two or more GLP-1 agonists selected from the GLP-1 agonists described herein.

The at least one GLP-1 agonist is further preferably independently selected from exendin-4, Pro³⁸exendin-4(1-39)-Lyse-NH₂, and Arga³⁴Lys²⁶ (N^{E}(γ-giutamyl(N^{α}-hexadecanoyl)))GLP-1(7-37) [liraglutide], and pharmacologically tolerable salts thereof.

The compositions of the invention contain the GLP-1 agonist in an amount of 10 μg/ml to 20 mg/ml, preferably 25 μg/ml to 15 mg/mL. For the acidic to neutrally dissolved GLP-1 agonists the figures are preferably 20 μg/ml to 300 μg/ml, and for the neutral to basic agonists they are preferably 500 μg/ml to 10 mg/mL. For exendin-4 analogs, 20 μg/ml to 150 μg/ml are preferred.

In the present specification the term “insulin” encompasses not only unmodified insulins but also insulin analogs, insulin derivatives, and insulin metabolites. The compositions of the invention comprise one or more independently selected from the group consisting of insulins (e.g., unmodified insulins), insulin analogs, insulin derivatives, and insulin metabolites, and any desired combinations thereof.

The at least one insulin may independently be selected from the group consisting of bovine insulins, analogs, derivatives, and metabolites thereof, porcine insulins, analogs, derivatives, and metabolites thereof, and human insulins, analogs, derivatives, and metabolites thereof. Preferably the at least one insulin is independently selected from human insulins, analogs, derivatives, and metabolites thereof.

Furthermore, an insulin of the invention may be selected independently from unmodified insulins, more particularly from bovine insulins, porcine insulins, and human insulins.

The at least one insulin may independently be selected from the group consisting of bovine insulins, porcine insulins, and human insulins. More preferably the at least one insulin is independently selected from human insulins. An insulin of the invention may be selected from unmodified insulins, more particularly from bovine insulins, porcine insulins, and human insulins.

Insulin derivatives of the invention are derivatives of a naturally occurring insulin and/or an insulin analog, which are obtained by chemical modification. The chemical modification may consist, for example, in the addition of one or more defined chemical groups onto one or more amino acids”

Insulin analogs which are described in EP 0 214 826, EP 0 375 437, EP 0 678 522, EP 0 885 961, EP 0 419 504, WO 92/00321, German patent applications 10 2008 003 568.8 and 10 2008 003 566.1, and EP-A 0 368 187 may be part of the compositions of the invention. The documents EP 0 214 826, EP 0 375 437, EP 0 678 522, EP 0 419 504, WO 92/00321, and EP-A 0 368 187 are included herein by reference.

One preferred insulin analog of the invention may be selected from the group consisting of Gly(A21)-Arg(B31)-Arg(B32) human insulin (insulin glargine, Lantus); Arg(A0)-His(A8)-Glu(A15)-Asp(A18)-Gly(A21)-Arg(B31)-Arg(B32) human insulin amide, Lys(B3)-Glu(B29) human insulin; Lys^{B28}Pro^{B29} human Insulin (insulin lyspro), B28 Asp human insulin (insulin aspart), human insulin in which proline in position B28 has been substituted by Asp, Lys, Leu, Val or Ala and where Lys in position B29 may be substituted by Pro; AlaB26 human insulin; des(B28-B30) human insulin; des(B27) human insulin or B29Lys(ε-tetradecanoyl),des(B30) human insulin (insulin detemir).

A preferred insulin derivative of the invention may be selected from the group consisting of B29-N-myristoyl-des(B30) human insulin, B29-N-palmitoyl-des(B30) human insulin, B29-N-myristoyl human insulin, B29-N-palmitoyl human insulin, B28-N-myristoyl Lys^{B28}Pro^{B29} human insulin, B28-N-palmitoyl-Lys^{B28}Pro^{B29} human insulin, B30-N-myristoyl-Thr^{B29}Lys^{B30} human Insulin, B30-N-palmitoyl-Thr^{B29}Lys^{B30} human insulin, B29-N—(N-palmitoyl-Y-glutamyl)-des(B30) human insulin, B29-N—(N-lithocholyl-Y-glutamyl)-des(B30) human insulin, B29-N-(ω-carboxyheptadecanoyl)-des(B30) human insulin, and B29-N-(ωw-carboxyheptadecanoyl) human insulin.

A more highly preferred insulin derivative of the invention is selected from the group consisting of Gly(A21)-Arg(B31)-Arg(B32) human insulin, Lys^{B28}Pro^{B29} human insulin-(insulin lyspro), B28 Asp human Insulin (insulin aspart), B29Lys(E tetradecanoyl),desB30 human insulin (insulin detemir).

The term “at least one insulin” includes combinations of the herein-described insulins, analogs, derivatives, and metabolites thereof which are used in the compositions of the invention, e.g., any desired combinations of two or more selected from the herein-described insulins, analogs, derivatives, and metabolites.

The compositions of the invention contain 60-6000 nmol/ml, preferably 240-3000 nmol/ml, of an insulin as defined herein. Depending on the insulin used, a concentration of 240-3000 nmol/ml corresponds approximately to a concentration of 1.4-35 mg/ml or 40-500 units/ml.

In the 2 to 10, preferably 3 to 5, pens cover all system, the compositions are in the range from 20 μg/ml of GLP-1 agonist and 100 U/ml of insulin to 300 μg/ml of GLP-1 agonist and 500 U/ml of insulin. Preference is given to the following concentration ranges: 25 μg/ml and 100 U/ml, 33 μg/ml and 100 U/ml, 40 μg/ml and 100 U/mI, 66 μg/ml and 100 U/ml, and 75 μg/ml and 100 U/ml.

The desired dosage range of the insulin is in particular a dosage with a synergistic effect. Here the values are 5 to 100 U, preferably 15 to 80 U. For the GLP-1 agonist the values for the dosage range are 5 μg to 2 mg, preferably 10 μg to 1.8 mg, more preferably 10 μg to 30 μg.

The preferred presentation form of the pharmaceutical compositions of the present invention is that of liquid compositions suitable in particular for parenteral administration, more preferably for injection, most preferably for subcutaneous injection. In particular the pharmaceutical composition of the present invention is suitable for injection once daily.

The pharmaceutical composition of the present invention may have an acidic or physiological pH. An acidic pH range is situated preferably in the range of pH 1-6.8, more preferably pH 3.5-6.8, even more preferably pH 3.5-4.5, most preferably at a pH about 4.0-4.5. A physiological pH is situated preferably in the range of pH 4.0-8.5, more preferably pH 5.0 to 8.5, even more preferably pH 6.0 to 8.5.

The composition of the invention may comprise a suitable preservative. Examples of suitable preservatives include phenol, m cresol, benzyl alcohol and/or p hydroxybenzoic esters.

The composition of the invention may further comprise a suitable buffer. Buffer substances which can be used, particularly for setting a pH level between about 4.0 and 8.5, include, for example, sodium acetate, sodium citrate, sodium phosphate, etc.

Otherwise, physiologically unobjectionable dilute acids (typically HCl) or alkalis (typically NaOH) are suitable for setting the pH level. Preferred concentrations of the buffers and also of corresponding salts are in the range of 5-250 mM, more preferably in the range of 10-100 mM.

The composition of the invention may comprise zinc ions. The concentration of the zinc ions is preferably in the range from 0 μg/ml to 500 μg/ml, more preferably from 5 μg to 200 μg of zinc/ml.

The composition of the invention may further comprise suitable isotonicity agents. Suitable examples include glycerol, dextrose, lactose, sorbitol, mannitol, glucose, NaCl, calcium compounds or magnesium compounds such as CaCl₂, etc. Glycerol, dextrose, lactose, sorbitol, mannitol, and glucose are typically in the range of 100-250 mM, NaCl in a concentration of up to 150 mM.

The composition of the invention may further comprise a surfactant. A surfactant may greatly increase the stability of acidic insulin compositions. Using surfactant it is even possible to prepare compositions which guarantee the superior stability with respect to hydrophobic aggregation nuclei over a number of months with temperature exposure.

The surfactant is preferably selected from the group consisting of partial and fatty acid esters and ethers of polyhydric alcohols such as of glycerol and of sorbitol, and polyols, the partial and fatty acid esters and ethers of glycerol and of sorbitol being selected from a group containing Span®, Tween®, Myrj®, Brij®, and Cremophor®; and the polyols being selected from the group of polypropylene glycols, polyethylene glycols, poloxamers, polysorbates, Pluronics, and Tetronics. Preferred concentrations of the surfactants are in the range of 5-200 μg/ml, preferably of 5-120 μg/ml and more preferably of 20-75 μg/ml.

The composition of the invention may further comprise other additives such as, for example, salts, which retard the release of at least one insulin.

One particularly preferred subject of the invention is a medicament as described herein comprising at least one insulin independently selected from Lys^{B28}Pro^{B29} human insulin (insulin lyspro), B28 Asp human Insulin (insulin aspart), B29Lys(e-tetradecanoyl),desB30 human insulin (insulin detemir), and insulin glargine (Gly(A21)-Arg(B31)-Arg(B32) human insulin), and comprising AVE0010 and/or a pharmacologically tolerable salt thereof. A further particularly preferred subject is a medicament as described herein comprising insulin glargine (Gly(A21)-Arg(B31)-Arg(B32) human insulin) and AVE0010 (des Pro³⁶exendin-4(1-39)-Lys₆-NH₂) and/or a pharmacologically tolerable salt thereof. The compositions of these particularly preferred medicaments preferably have an acidic pH of 1-6.8, more preferably pH 3.5-6.8, even more preferably pH 3.5-5.0, most preferably a pH of about 4.0 to 4.5. In addition, the compositions of these particularly preferred medicaments may comprise a surfactant as described herein.

A further subject of the invention is a combination of insulin glargine (Gly(A21)-Arg(B31)-Arg(B32) human insulin) and AVE0010 (des Pro³⁶exendin-4(1-39)-Lys₆-NH₂) and/or a pharmacologically tolerable salt thereof.

The invention further provides a method of treating a patient with a kit or medicament of the invention as described herein.

The method of the invention for treating a patient comprises the administration of a medicament of the invention comprising at least one insulin and at least one GLP-1 agonist, the medicament being formulated and/or compounded such that it contains the insulin and the GLP-1 agonist each in a predetermined amount and can be administered in a dose adapted to the individual requirement of a patient.

More particularly the method comprises the administration of a medicament comprising a first pharmaceutical composition and a second pharmaceutical composition, and, optionally, at least one further pharmaceutical composition, each comprising at least one insulin and at least one GLP-1 agonist, and comprising the at least one insulin and/or the at least one GLP-1 agonist in different weight fractions relative to the total weight of the composition, said method comprising:
(a) selecting a dose of the at least one insulin that is to be administered,
(b) selecting a dose of the at least one GLP-1 agonist that is to be administered,
(c) selecting a composition, from the first, second, and, where used, at least one further compositions of the medicament that comprises the doses from (a) and (b) in a concentration such that the doses from (a) and (b) are present in the same volume, and
(d) determining and administering an amount which corresponds to the doses from (a) and (b).

The dose according to step (a) and/or step (b) is determined according to the individual requirement of the patients.

Step (c) of the treatment method of the invention can be carried out on the basis of a table. This table may be part of the medicament of the invention. Example 11 contains an example of a table of the invention.

The method of treating a patient may more particularly comprise the administration a medicament, the medicament comprising a first pharmaceutical composition and a second pharmaceutical composition, and, optionally, at least one further pharmaceutical composition, the first pharmaceutical composition comprising at least one first active compound, and the second pharmaceutical composition comprising at least one first active compound and at least one second active compound, the at least one further pharmaceutical composition comprising at least one first active compound and at least one further active compound, and the method comprising the following steps:
(i) selecting a dose of the at least one first active compound that is to be administered, and determining the total amount of the first, second, and, where used, at least one further composition, so that the selected dose of the at least one first active compound is present in the total amount,
(ii) selecting a dose of the at least one second active compound that is to be administered and determining the amount of the second composition so that the selected dose of the at least one second active compound is present in the amount of the second composition,
(iii) where appropriate, selecting a dose of the at least one further active compound that is to be administered, and determining the amount of the at least one further composition so that the selected dose of the at least one further active compound is present in the amount of the at least one further composition,
(iv) administering an amount of the first composition to the patient, the administered amount corresponding to the total amount as per step (i) minus the amount of the second composition as per step (ii), and, where appropriate, minus the amount of the at least one further composition as per step (iii), and
(v) administering the amount of the second composition that was determined in step (ii), and, where appropriate, the amount of the at least one further composition that was determined in step (iii), to the patient

The first active compound may be an insulin, and the second active compound may be a GLP-1 agonist. Hence the method of treating a patient may comprise more particularly the administration of a medicament, the medicament comprising a first pharmaceutical composition and a second pharmaceutical composition, and, optionally, at least one further pharmaceutical composition, the first pharmaceutical composition comprising at least one Insulin, and the second pharmaceutical composition comprising at least one insulin and at least one GLP-1 agonist, and the at least one further pharmaceutical composition comprising at least one insulin and at least one further active compound, and the method comprising the steps of:
(i) selecting a dose of the at least one insulin that is to be administered, and determining the total amount of the first, second, and, where used, at least one further composition, so that the selected dose of the at least one insulin is present in the total amount,
(ii) selecting a dose of the at least one GLP-1 agonist that is to be administered and determining the amount of the second composition so that the selected dose of the at least one GLP-1 agonist is present in the amount of the second composition,
(ill) where appropriate, selecting a dose of the at least one further active compound that is to be administered, and determining the amount of the at least one further composition so that the selected dose of the at least one further active compound is present in the amount of the at least one further composition,
(iv) administering an amount of the first composition to the patient, the administered amount corresponding to the total amount as per step (i) minus the amount of the second composition as per step (ii), and, where appropriate, minus the amount of the at least one further composition as per step (iii), and
(v) administering the amount of the second composition that was determined in step (ii), and, where appropriate, the amount of the at least one further composition that was determined in step (iii), to the patient.

The first active compound may be a GLP-1 agonist, and the second active compound may be an insulin. Hence the method of treating a patient may comprise more particularly the administration of a medicament, the medicament comprising a first pharmaceutical composition and a second pharmaceutical composition, and, optionally, at least one further pharmaceutical composition, the first pharmaceutical composition comprising at least one GLP-1 agonist, and the second pharmaceutical composition comprising at least one GLP-1 agonist and at least one insulin, and the at least one further pharmaceutical composition comprising at least one GLP-1 agonist and at least one further active compound, and the method comprising the steps of:
(I) selecting a dose of the at least one GLP-1 agonist that is to be administered, and determining the total amount of the first, second, and, where used, at least one further composition, so that the selected dose of the at least one GLP-1 agonist is present in the total amount,
(ii) selecting a dose of the at least one insulin that is to be administered and determining the amount of the second composition so that the selected dose of the at least one insulin is present in the amount of the second composition,
(iii) where appropriate, selecting a dose of the at least one further active compound that is to be administered, and determining the amount of the at least one further composition so that the selected dose of the at least one further active compound is present in the amount of the at least one further composition,
(iv) administering an amount of the first composition to the patient, the administered amount corresponding to the total amount as per step (i) minus the amount of the second composition as per step (ii), and, where appropriate, minus the amount of the at least one further composition as per step (iii), and
(v) administering the amount of the second composition that was determined in step (ii), and, where appropriate, the amount of the at least one further composition that was determined in step (iii), to the patient

Steps (i), (ii) and/or (iii) may be carried out on the basis of at least one table, which may be part of the medicament For each of steps (i), (ii), and (iii) independently of one another a table may be provided.

The treatment method of the invention may be used more particularly for treating patients with diabetes, more particularly with diabetes type 1 or II. Preferably the method is used to adjust the fasting, postprandial and/or postabsorptive blood glucose concentration, for improving glucose tolerance, for preventing hypoglycemia, for 2.0 preventing loss of function of the pancreatic cells, for weight loss and/or for preventing weight gain.

The invention further provides a method of preparing a medicament of the invention, comprising formulating and/or compounding, such that it contains the insulin and the GLP-1 agonist each in a predetermined amount and can be administered in a dose adapted to the individual requirement of a patient in the preparation method the medicament is preferably formulated and compounded such that one of the herein-described medicaments of the invention can be obtained, as for example a medicament of the invention comprising a first pharmaceutical composition and a second pharmaceutical composition, and, optionally, at least one further pharmaceutical composition, each comprising at least one insulin and at least one GLP-1 agonist, and comprising the at least one insulin and/or the at least one GLP-1 agonist in different weight fractions relative to the total weight of the composition.

The invention is illustrated by the following figures and the following examples, which do not restrict the invention in any way whatsoever.

### DETAILED DESCRIPTION OF THE DRAWINGS

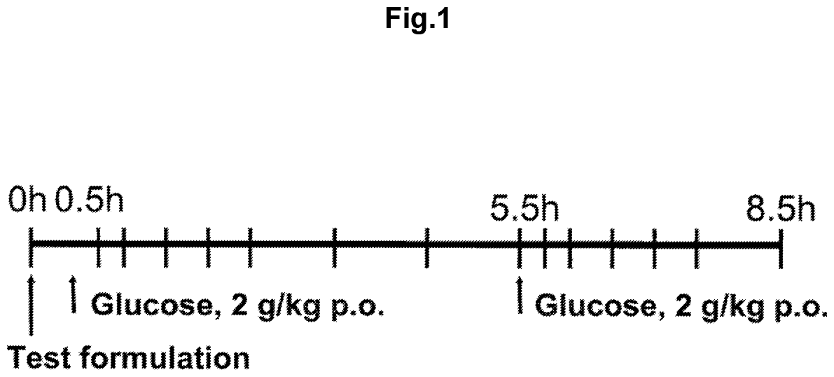

FIG. 1: Study design for oral glucose tolerance test

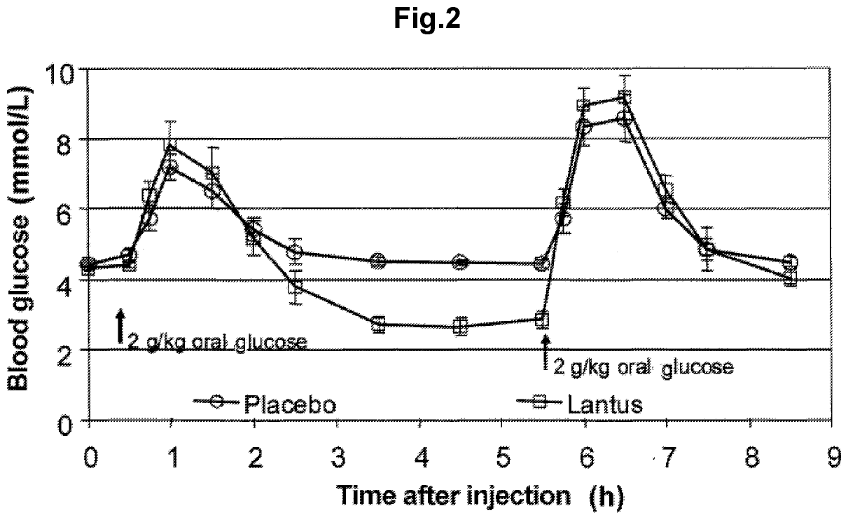

FIG. 2: OGTT in the dog: Effect of insulin glargine relative to placebo.

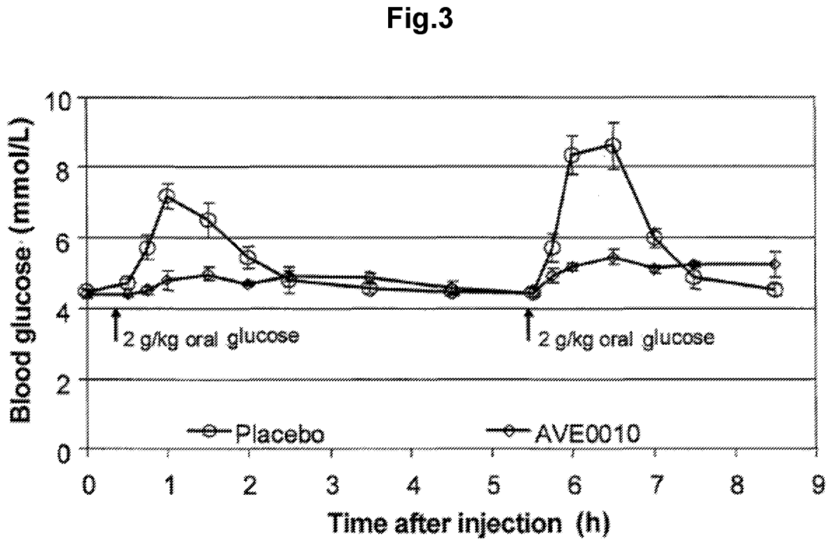

FIG. 3: OGTT in the dog: Effect of AVE0010 relative to placebo.

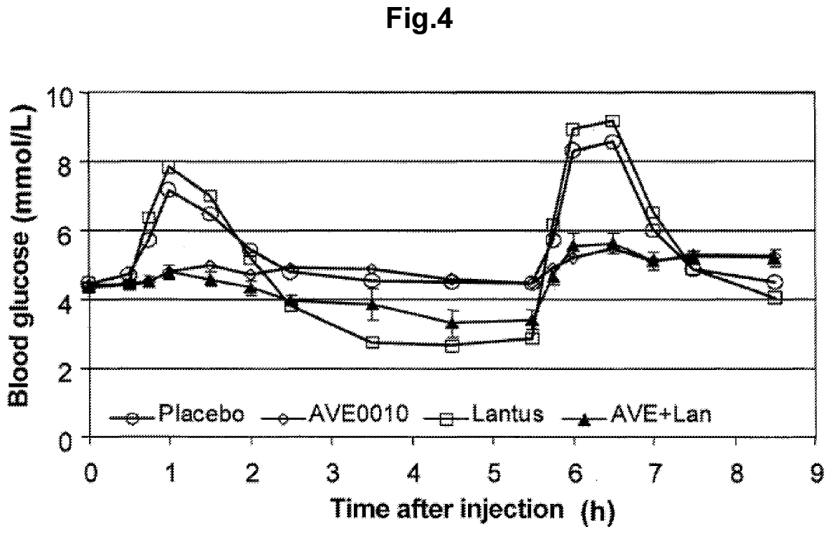

FIG. 4: OGTT in the dog: Effect of an AVE0010/insulin glargine combination on blood glucose level.

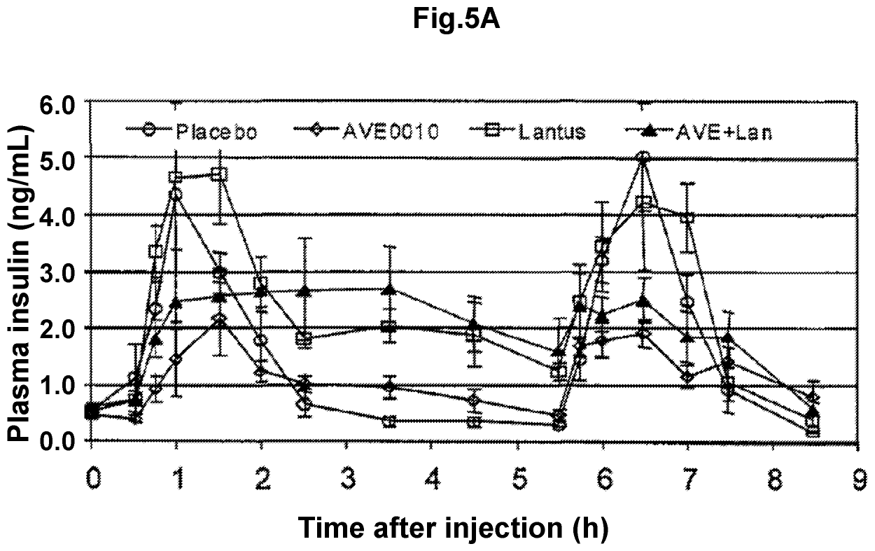

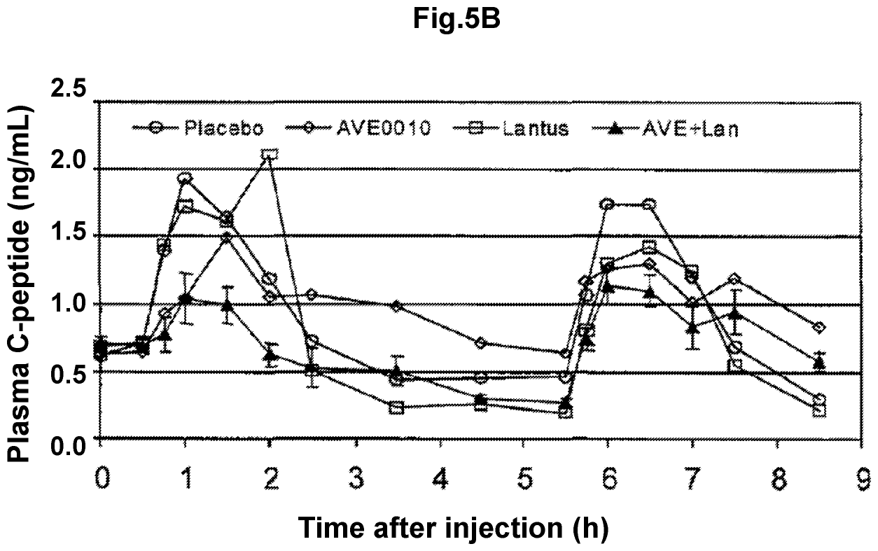

FIG. 5A: OGTT in the dog: Effect of an AVE0010/insulin glargine combination on plasma insulin.

FIG. 5B: OGTT in the dog: Effect of an AVE0010/insulin glargine combination on the c-peptide level.

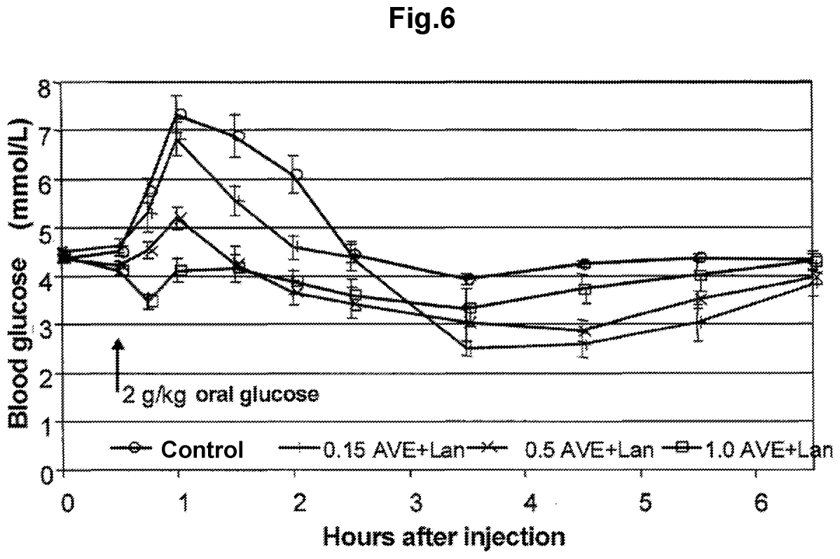

FIG. 6: OGTT in the dog: Effect of a dose lowering of AVE0010 with different proportions relative to insulin glargine in the combined formulation.

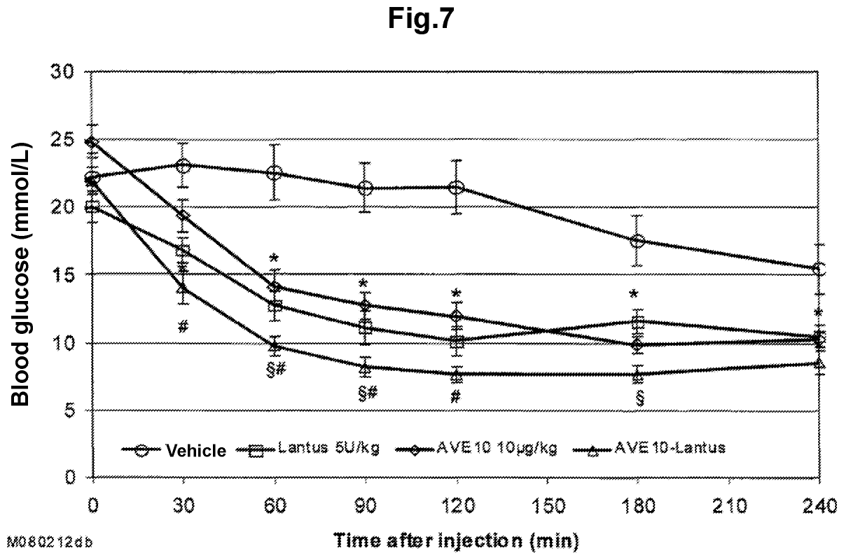

FIG. 7: Effect of an AVE0010/insulin glargine combination on blood glucose in the diabetic db/db mouse.

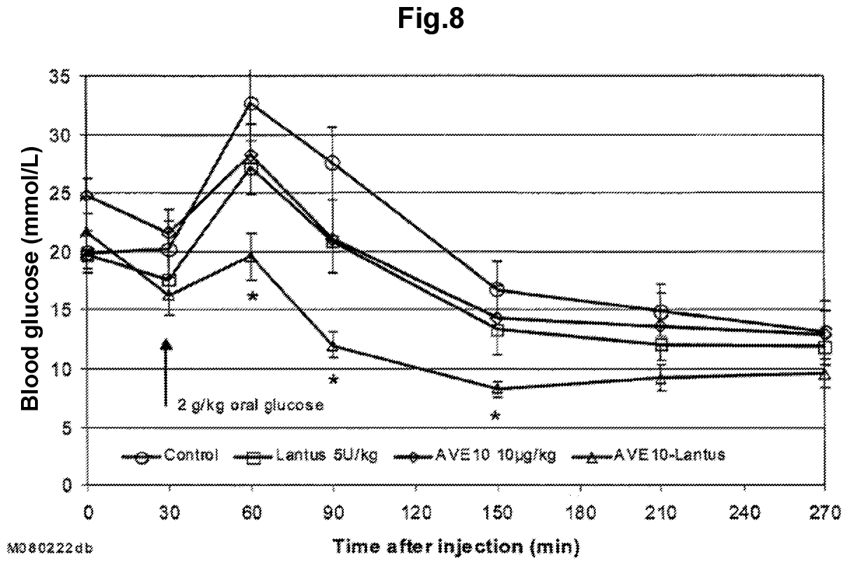

FIG. 8: Effect of an AVE0010/insulin glargine combination in the oral glucose tolerance test in the diabetic db/db mouse.

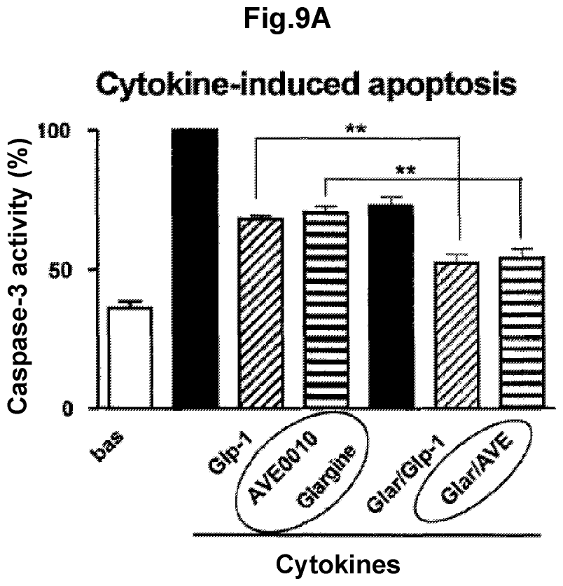

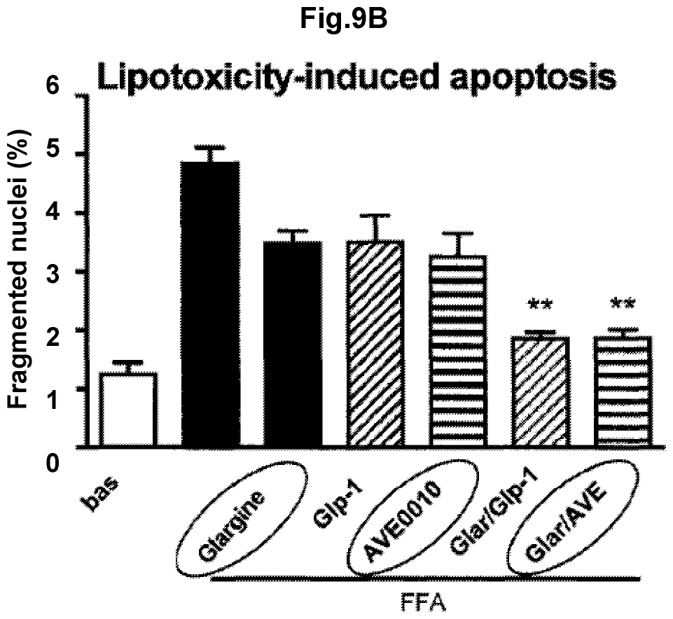

FIG. 9A: Effect of an AVE0010/insulin glargine combination on cytokine-induced-cell apoptosis in vitro.

FIG. 9B: Effect of an AVE0010/insulin glargine combination on lipotoxicity-induced cell apoptosis in vitro.

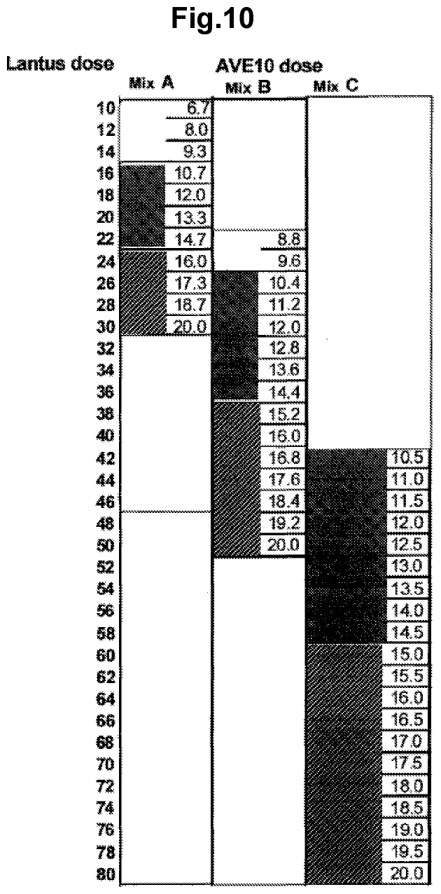

FIG. 10: The “3 pens cover all” system.

### EXAMPLES

### Example 1

Model: Oral glucose tolerance test (OGTT) in healthy dogs: Comparison of the insulin glargine/AVE0010 combination with the two individual active compounds.

Animals
Male normoglycemic beagles
Bodyweight: ˜15 kg
Number per group: n=6

Study Design (see FIG. 1)
Individual subcutaneous injections of placebo or test formulation at time 0
2 oral administrations of glucose, at 2 g of glucose/kg of bodyweight, at times 30 min and 5 h
Blood samples are taken to determine blood glucose, plasma insulin, and c-peptide

Group Division (n 6)
Placebo (Lantus placebo formulation without API)
Insulin glargine (0.3 IU/kg s.c., equivalent to 1.8 nmol/kg). Insulin glargine is Gly(A21)-Arg(B31)-Arg(B32) human insulin.
AVE0010 (10 μg/kg s.c. in Lantus placebo formulation, equivalent to 2 nmol/kg). AVE0010 is des Pro 36exendin-4(1-39)-Lys₆-NH₂.
AVE0010/insulin glargine combination (10 μg/kg AVE0010/0.3 IU/kg insulin glargine s.c.)

### Example 2

OGTT in the Dog: Effect of insulin Glargine Relative to Placebo

The experiment was carried out in accordance with the protocol described in example 1.
repeated OGTT (2 g/kg p.o.)
male beagle, n=6
mean±Sem
placebo=Lantus placebo
insulin glargine (0.3 U/kg s.c.)

Result: The data are shown in FIG. 2. The single administration of insulin glargine does not prevent the OGTT-induced increase in blood glucose. Insulin glargine reinforces the expected delayed lowering of blood glucose concentration in the postabsorptive phase.

### Example 3

OGTT in the dog: Effect of AVE0010 relative to placebo

The experiment was carried out in accordance with the protocol described in example 1.
repeated OGTT (2 g/kg p.o.)
male beagle, n=6
mean±Sem
placebo=Lantus placebo
AVE0010 (10 μg/kg s.c.)

Result: The data are shown in FIG. 3. AVE0010 prevents the OGTT-induced postprandial increase in blood glucose almost completely. There is no effect on the glucose concentration in the postabsorptive phase. This example shows that the effect of AVE0010 on the OGTT-induced postprandial increase in blood glucose is complementary to the blood sugar-lowering effect of insulin glargine in the postabsorptive phase.

### Example 4

OGTT in the dog: Effect of an AVE0010/insulin glargine combination on the blood glucose level.

The experiment was carried out in accordance with the protocol described in example 1.
repeated OGTT (2 g/kg p.o.)
male beagle, n=6
mean±Sem
placebo=Lantus placebo
AVE0010 (10 μg/kg s.c.)
Insulin glargine (0.3 U/kg s.c.)
AVE+Lan (=premix of 10 μg/kg of AVE0010 and 0.3 U/kg of insulin glargine in one formulation)

Result: The data are shown in FIG. 4. The combination has the same action on the postprandial glucose increase as AVE0010 (cf. example 3). The hypoglycemiceffect of insulin glargine in the postabsorptive phase is likewise present, but attenuated (cf. example 2). This is a synergistic effect of insulin glargine and AVE0010, since AVE0010 alone has no effect on the level of glucose, which has fallen again following administration of glucose, and insulin glargine on its own has no effect on the postprandial glucose level.

### Example 5

OGTT in the dog: Effect of an AVE0010/insulin glargine combination on the plasma insulin and the c-peptide level.

The experiment was carried out in accordance with the protocol described in example 1.
repeated OGTT (2 g/kg p.o.)
male beagle, n=6
mean±Sem
placebo=Lantus placebo
AVE0010 (10 μg/kg s.c.)
Insulin glargine (0.3 U/kg s.c.)
AVE+Lan (=premix of 10 μg/kg of AVE0010 and 0.3 U/kg of insulin glargine in one formulation)

The C-peptide is released in the course of the conversion of proinsulin to insulin, and serves as a marker for the secretion of insulin by the pancreatic β-cells. In a glucose loading test, the c-peptide can be used to determine the response capacity of the pancreas.

Result: The data are shown in FIG. 5A and FIG. 5B. In the combination group, the postprandial reduction in insulin is followed by an increased postabsorptive insulin glargine level C-peptide levels for the combination correspond to the insulin curve of AVE0010 during the prandial phases, and of insulin glargine during the postabsorptive phase.

### Example 6

OGTT in the dog: Effect of a dose lowering of AVE0010 with different proportions to insulin glargine in the combined formulation.

The experiment was carried out in accordance with the protocol described in example 1.
repeated OGTT (2 g/kg p.o.)
male beagle, n=11/6/6/6
mean±Sem
control Lantus placebo
AVE+Lan (=premix of 0.15 to 1.0 μg/kg of AVE0010 and 0.3 U/kg of insulin glargine in one formulation). In examples 2 to 5, AVE0010 concentrations of 10 μg/kg were used.

Result: The data are shown in FIG. 6. A reduction in the AVE0010 dose from 10 μg/kg (cf. in particular example 4) to 1 μg/kg (i.e., by a factor of 10), and the resultant increase in the proportion of insulin glargine to AVE0010, has no effect on the synergistic activity of the combination of AVE0010 with insulin glargine (cf. in particular example 4). Only at significantly smaller AVE0010 doses does the effect of the combination approach the effect of insulin glargine alone (et in particular FIG. 2). The AVE0010 dose may therefore be varied at least within one order of magnitude (i.e., by a factor of at least 10) without loss of the synergistic effect.

### Example 7

Model: Diabetic, insulin-resistant db/db mouse: Comparison of the insulin glargine/AVE0010 combination with the two individual active compounds.

Animals
Female db/db mouse
Age: 10-11 weeks
Number per group: n=10

Study Design
Individual subcutaneous injection of placebo or test formulation
Taking of blood samples to determine blood glucose

Group Division
Placebo (=Lantus placebo formulation without API)
AVE0010 (10 μg/kg s.c.)
Insulin glargine (5 IU/kg s.c.)
AVE0010/insulin glargine combination (premix of 10 μg/kg of AVE0010 plus 5 IU/kg of insulin glargine s.c.)

### Example 8

Effect of an AVE0010/insulin glargine combination on blood glucose in the diabetic db/db mouse

The experiment was carried out in accordance with the protocol described in example 7.
Female db/db mouse, 10 weeks
n=10, mean±Sem
Vehicle=Lantus placebo
AVE0010 (10 μg/kg sc)
Lantus (5 U/kg sc)
AVE0010/insulin glargine (=premix of AVE0010 10 μg/kg and insulin glargine 5 U/kg in one formulation)

Result: The data are shown in FIG. 7. In diabetic db/db mice, the AVE0010/insulin glargine combination produced a more rapid and more pronounced decrease in the blood glucose concentration as compared with the two individual active compounds. Consequently the combination takes diabetic db/db mice closer to normoglycemia than either of the two active compounds alone.

### Example 9

Effect of an AVE0010/insulin glargine combination in the oral glucose tolerance test in the diabetic db/db mouse

The experiment was carried out in accordance with the protocol described in example 7. Additionally an OGTT (2 g/kg p.o. @ 30 min) was carried out.
Female db/db mouse, 11 weeks
n=10, mean±Sem
Control=Lantus placebo
AVE0010 (10 μg/kg sc)
Insulin glargine (5 U/kg sc)
AVE0010/insulin glargine (=premix of AVE0010 10 μg/kg and insulin glargine 5 U/kg in one formulation)

Result The data are shown in FIG. 8. The AVE0010/insulin glargine combination leads to significantly improved glucose tolerance and lower postabsorptive glucose levels.

### Example 10

Effect of the AVE0010/insulin glargine combination on cytokine- and lipotoxicity-induced β-cell apoptosis in vitro.
Insulinoma cell line INS-1, rat
Incubation with the test compound for 5 h
Further incubation with a cytokine mix for 22 h (1 ng/mL IFN-γ+4 ng/mL IL-13) or
Further incubation with 0.5 mM FFA for 18 h (palmitates: BSA 3:1)

The measures used for the apoptosis are the caspase-3 activity and the fragmentation of the cell nuclei, which correlate with apoptosis.

Result: The data are shown in FIG. 9A and FIG. 9B. AVE0010 or insulin glargine (glargine, Glar) alone prevent the apoptosis by ˜40-50%. The AVE0010 and insulin glargine combination prevents apoptosis significantly better. On the basis of this synergistic effect, the combination brings about increased protection against cytokine- and lipotoxicity-induced apoptosis.

### Example 11

The “3 pens cover all” system (FIG. 10)
3 premix pens with 3 different predetermined proportions:
   Mix A: 100 U of insulin glargine+66.66 μg of AVE0010 per mL
   Mix B: 100 U of insulin glargine+40 μg of AVE0010 per mL
   Mix C: 100 U of insulin glargine+25 μg of AVE0010 per mL.
Use of the 3 premix pens: The table in FIG. 10, representing an example, starts from a therapeutic range of 15 to 80 U per dose of insulin glargine and 10 to 20 μg of AVE0010. For a particular patient, a dose of insulin glargine to be administered is specified or predetermined. The predetermined dose is looked up in the left-hand column. Where the columns MIX A-MIX C specify a corresponding AVE0010 dose in the range between 10 and 20 μg, the corresponding MIX is selected, dosed, and administered. The ranges are overlapping: for example, in the case of a requirement of 26 to 30 U of insulin glargine, it will be possible to choose MIX A or MIX B (with a higher dose of AVE0010). The same applies to MIX B and C. If, for example, a dose of 50 U of insulin is intended, then 0.5 ml of MIX B or MIX C can be dosed. This dose contains 20 μg (MIX B) or 12.5 μg (MIX C) of AVE0010.
Conclusion: On the assumption that a probable AVE0010 effect is obtained at between 10 and 15 μg, and a therapeutic effect between 15 and 22 μg, almost all patients who take insulin glargine doses of 15-80 U can also obtain therapeutic doses of AVE0010 of between 10 and 20 μg if they use one of the three premix pens which contain three different insulin glargine:AVE0010 ratios (Mix A, B or C). On the basis of the broad range of possible proportions of insulin glargine to AVE0010 (cf. example 6) with a synergistic effect, the proportions in the pens can be tailored such that for each dose of insulin glargine there is a synergistic dose of AVE0010 in at least one pen.

### Example 12

This example shows how a combination of two or more active compounds can be formulated in such a way that, when two or more compositions are combined, both active compounds can be administered in any desired amounts and in any desired proportions to one another. It is taken into account here that at least one of the active compounds must not be diluted as a result of the combining (e.g., through mixing directly prior to administration).

In this example, the designations “active A” and “active B” stand for any desired active compounds. In particular, active A is an insulin and active B is a GLP-1 agonist. Active A can also be a GLP-1 agonist, and active B can also be an insulin.

### 1. Comparative Example

For a combination therapy with an active A (e.g., an insulin) and an active B (e.g., a GLP-1 agonist), a container 1 with a composition with active A at a concentration of a mg/ml, and a container 2 with a composition with active B at a concentration of b mg/ml, are provided.

For the administration of a combination of the two actives, a volume V₁ ml from container 1 and a volume V₂ ml from container 2 are mixed.

For the dosing of the two actives, at given concentrations a and b, the volumes V₁ and V₂ to be administered are selected in dependence on the amount of the actives A and B to be administered. The volumes V₁ and V₂ of the two actives are determined on the basis of the amount of active, as follows:

V₁·a mg Amount of active A:

V₂·b mg Amount of active B:

The concentrations of the actives A and B in the mixture of the two compositions are determined as follows.

x mg/mL=V₁·a/(V₁+V₂) Active A:

y mg/mL=V₂·b/(V₁+V₂) Active B:

V₁+V₂ is the total administered volume. This means that the two actives dilute one another. With this system, therefore, it is not possible to keep, for example, the concentration of the active A (e.g., of the insulin) at a predetermined level in the case of varying amounts of active B.

### 2. Inventive Example

In this example, for a combination therapy with an active A (e.g., an insulin) and an active B (e.g., a GLP-1 agonist), a container 1 with a composition with active A at a concentration of a mg/ml, and a container 2 with a composition with active A at a concentration of a mg/ml and with active B at a concentration of b mg/ml, are provided. The concentration of the active A is therefore the same in both compositions.

For the administration of a combination of the two actives, a volume V₃ ml from container 1 and a volume V₂ ml from container 2 are mixed.

For the dosing of the two actives, at given concentrations a and b, the volumes V₃ and V₂ to be administered are selected in dependence on the amount of the actives A and B to be administered. The volumes V₃ and V₂ of the two actives are determined on the basis of the amount of active, as follows:

(V₃·a+V₂)·a(mg)) Amount of active A:

V₂·b mg Amount of active B:

The concentrations of the actives A and B are determined as follows.

a mg/mL=(V₃·a+V₂·a)/(V₃+V₂) Active A:

z mg/mL=V₂·b/(V₃+V₂) Active B:

V₃+V₂ is the total administered volume. From the above calculation it is evident that the concentration of the active A is always a mg/ml, i.e., is constant, irrespective of what volume ratio V₃/V₂ is being dosed.

Comparing the comparative example (see section 1) with the present inventive example, it is apparent that, for an equal dosing quantity of actives A and B, the total volume required in the inventive example is lower.

For a given dose (amount of active compound) of the active A, the figure in the comparative example is: V₁·a mg

In the inventive example it is: (V₃·a+V₂·a) mg

Since the amount of active compound is to be the same in both cases,

(V₃·a+V₂·a)=V₁·a

(V₃+V₂)·a=V₁·a

and V₃+V₂=V₁

or V₃=V₁−V₂

Here, the volume V₂ in which the active B is administered is the same in both cases.

The total volume in the comparative example is V₁+V₂

The total volume in the inventive example is V₃+V₂

According to the above equation, for the inventive example it is the case that:

V₃+V₂=V₁−V₂+V₂=V₁

This volume V₁ is smaller than the volume V₁+V₂ of the comparative example.

As a result of the mixing of the composition with actives A and B with the composition with active A, active B is diluted. This dilution is less than the dilution of the active B in the comparative example (i.e., the concentration b>concentration z>concentration Y):

b>z

b>V₂·b/(V₃+V₂)

b>b·V₂/M(V₃+V₂), where V₂/(V₃+V₂) is <1, and

z>y

V₂·b/(V₃+V₂)>V₂·b/(V₁+V₂)

1/(V₃+V₂)>1/(V₁+V₂)

1/(V₁−V₂+V₂)>1/(V₁+V₂)

1/V₁>1/(V₁+V₂)

Hence the dosing system of the invention for administering variable doses of the actives A (e.g., an insulin) and B (e.g., a GLP-1 agonist) has three advantages over the comparative system:
The concentration of active A (e.g., an insulin) can be kept constant at a predetermined level
Where the doses of actives A and B to be administered are the same, the total volume to be administered is smaller.
The dilution of active B (e.g., the GLP-1 agonist) is less than in the comparative experiment. Accordingly the concentration of active B can be held more easily within a predetermined range.

The present example can be readily extended to medicaments with three or more active compounds, the first active compound being present in all of the compositions (preferably in equal weight fractions) and there being at least one further active compound in each further composition. The first composition can be mixed with each further composition in the same proportion without the concentration of the active compound in the first composition becoming diluted.

1. A pharmaceutical composition comprising at least one insulin and at least one GLP-1 agonist, or medically acceptable salts thereof, wherein said pharmaceutical composition comprises the insulin and the GLP-1 agonist each in a predetermined amount, and is self-administered in a dose adapted to the individual requirement of a patient.

2. A method for treating diabetes mellitus in a patient in need thereof comprising administering to said patient a therapeutically effective amount of the pharmaceutical composition of claim 1.

3. The method of claim 2, wherein said patient is treated for type 1 or type 2 diabetes mellitus.

4. The method of claim 3, wherein the patient is previously or concurrently being treated for diabetic retinopathy.

5. The method of claim 2, wherein blood glucose is improved in the patient.

6. The method of claim 2, wherein the therapeutically effective amount of the pharmaceutical composition is administered by an automatic injection apparatus.

7. The method of claim 2, wherein the therapeutically effective amount of the pharmaceutical composition is administered once daily.

8. The method of claim 2, wherein said patient is further treated for weight loss and/or preventing weight gain.

9. A method of treatment for weight loss and/or preventing weight gain in a patient in need thereof comprising administering to said patient a therapeutically effective amount of the pharmaceutical composition of claim 1.

10. The method of claim 9, wherein the treatment for weight loss and/or preventing weight gain is for treatment of obesity.

11. The method of claim 9, wherein the therapeutically effective amount of the pharmaceutical composition is administered by an automatic injection apparatus.

12. The method of claim 9, wherein the therapeutically effective amount of the pharmaceutical composition is administered once daily.

13. A device comprising the pharmaceutical composition of claim 1, wherein the first and second compositions are in separate containers to allow the dosing of the compositions independently of one another.
